(19) United States
(12) Patent Application Publication
(10) Pub. No.: US 2025/0268742 A1
(43) Pub. Date: Aug. 28, 2025

(54) URINE MANAGEMENT SYSTEMS AND METHODS

(71) Applicant: Medline Industries, LP, Northfield, IL (US)

(72) Inventors: Amit Kumar Sharma, New Delhi (IN); Nishith Chasmawala, Gujarat (IN)

(21) Appl. No.: 18/856,505

(22) PCT Filed: Apr. 11, 2023
(86) PCT No.: PCT/IB2023/053679

§ 371 Date: Oct. 11, 2024

Related U.S. Application Data
(60) Provisional application No. 63/330,298, filed on Apr. 12, 2022.

(51) Int. Cl. A61F 5/44; A61F 5/451; A61M 1/00

(52) U.S. Cl. CPC A61F 5/4405; A61F 5/451; A61M 1/74; A61M 1/782; A61M 2202/0496; A61M 2205/3317; A61M 2205/3327; A61M 2205/3337; A61M 2205/3389

(57) ABSTRACT

Fluid (e.g., urine) management methods and apparatuses are described herein. The fluid management apparatuses may include a vacuum pump, a collection canister, and a collection bag. Negative pressure may be provided to the collection canister by the vacuum pump. The negative pressure may draw fluid into, as well as hold the fluid in, the collection canister. A controller may release the negative pressure from the collection canister to enable the fluid to flow from the collection canister into the collection bag.

### CLAIM OF PRIORITY

This patent application claims priority to U.S. provisional patent application No. 63/330,298, titled “URINE MANAGEMENT SYSTEMS AND METHODS” and filed on Apr. 12, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Urinary incontinence refers to the inability to control the release of the urine thereby causing involuntary leakage. The causes of urinary incontinence are varied and can range from stress urinary incontinence, overactive bladder, overflow incontinence and more. In some cases, a patient may be unable to attend to normal or conventional voiding of the bladder because the patient is bedridden, weak, or unconscious.

Proper collection of urine may be important for the care and well-being of the patient. Improper urine collection may result in infections including urinary tract infections which may adversely affect the health of the patients, particularly elderly patients. For example, extended use of transurethral catheter may increase the risk of bacterial infections as well as require periodic replacement.

Therefore, there is a need for an effective, external urine collection device.

### SUMMARY OF THE DISCLOSURE

Described herein are liquid (e.g., urine) collection management methods and apparatuses (e.g., devices, system, etc.). The apparatuses described herein may collect fluids in general, and urine in particular. The methods and apparatuses may use negative pressure to draw urine or liquid into a first canister or reservoir. In some examples, after the first canister exceeds a threshold, the urine or liquid may be transferred into a second canister or collection bag. The second canister or collection bag may be transparent or translucent and include external markings that allow a user or clinician to measure the collected fluids.

Any of the systems described herein may include a connecting tube, a collection unit, and a suction unit. The collection unit may include a collection canister coupled to the collection tube and a collection bag coupled to the collection canister. The suction unit may include a vacuum pump removably or permanently coupled to the collection unit and configured to provide a negative pressure to the collection canister and a controller configured to operate the vacuum pump based at least in part on a level of fluid in the collection canister, sequence of operation and whether the suction unit is coupled to the collection unit.

In any of the systems described herein, the collection unit may be configured to provide the negative pressure to the connecting tube and collect liquid (e.g., urine) from the connecting tube in the collection canister. Any of these apparatuses (e.g., systems) may include a valving sub-system, which may be adapted for use with and/or may be integrated into the suction pump and canister, and/or the tubing connecting any of these components. The valving sub-system may be configured to prevent the flow of fluid (e.g., urine) into the suction pump portion of the apparatus. In particular, the valving sub-system may also be configured to limit or prevent leakage and/or backflow that may otherwise result due to siphoning, even when suction has been stopped. For example, the valving sub-system may include a 3 way valve (e.g., a 3-way solenoid valve) connecting the outlet of the suction pump to atmosphere, and to suction pump bypass line, including a one-way valve, coupled to the suction port of the canister.

In any of the systems described herein, the collection bag may be coupled to the collection canister through a one-way valve configured to allow fluid flow from the collection canister to the collection bag and prohibit flow from the collection bag to the collection canister. Furthermore, in some examples, the collection unit may further include mounting pegs configured to detachably couple with attachment holes in the collection bag. In some other examples, the collection bag may be permanently coupled to the collection canister.

In any of the systems described herein the suction unit may include a proximity detector configured to determine whether the suction unit is coupled to the collection unit. In any of the systems described herein the collection canister may include a fluid level detector configured to determine a fluid level within the collection canister. Any appropriate fluid level detector may be used, including optical detectors (e.g., sensors), mechanical fluid level sensors, electrical fluid level sensors, magnetic fluid level sensors, etc.

In any of the systems described herein, the suction unit may include a vacuum release valve configured to release vacuum from the collection canister. In some examples, the controller may be configured to operate the vacuum release valve based on a detected level of fluid in the collection canister.

In some examples, the collection canister may be configured to release, by gravity, fluid into the collection bag when vacuum is released from the collection canister. The collection canister may include a drain valve configured to drain fluid from the collection bag while attached to the collection unit. Furthermore, in some examples the collection bag may include markings configured to indicate a quantity of fluid collected from the patient. As mentioned above, in any of these examples the fluid collection bag may be coupled to the collection canister by a valve the prevents flow from the collection canister into the collection bag when the pressure in the canister is less than the pressure in the bag. For example, the valve may be a flutter valve that is arranged so that it passes fluid from the collection canister into the collection bag only when the pressure in the collection canister is approximately the same as or is greater than the pressure within the collection bag. Thus, when vacuum is applied to the collection canister during operation of the apparatus the resulting negative pressure within the collection canister prevents the flutter valve from opening and transferring fluid from the canister into the collection bag, e.g., by gravity. After the suction is turned off, and pressure within the canister returns to atmosphere pressure, the flutter valve may open, allowing gravity to draw fluid from the collection canister into the collection bag.

In any of the systems described herein, the suction unit may include a battery configured to power the controller and the vacuum pump. In some examples, the suction unit may be configured to be powered by an external power source.

In any of the systems described herein, the suction unit may include an input/output interface configured to provide an operating status indication to the user. In some examples, the suction unit may be configured to release vacuum from the collection canister when the collection unit is detached from the suction unit. The connecting tube may be configured to be coupled to at least one of plurality of external urinary catheters.

Any of the methods described herein may include providing, by a vacuum pump, a negative pressure to a rigid collection canister, collecting, in response to the negative pressure, liquid (e.g., urine) from a connecting tube coupled to the rigid collection canister, where the liquid is collected in the rigid collection canister, and releasing the negative pressure from the rigid collection canister, where the release of the negative pressure causes liquid to flow from the rigid collection canister to a collection bag.

In any of the methods described herein, the negative pressure may be released by a vacuum release valve. In some examples, the vacuum release valve may be operated in response to a detected fluid level in the rigid collection canister. Furthermore, in some examples, the vacuum release valve may be operated by a controller.

In any of the methods described herein, liquid may flow through a one-way valve from the rigid collection canister to the collection bag.

Any of the systems described herein may include a suction unit that includes a collection canister coupled to a connecting tube and a vacuum pump coupled to the collection canister and configured to provide a negative pressure to the collection canister. The system may also include a collection bag detachably coupled to the collection canister and a controller configured to operate the vacuum pump and release the negative pressure based at least in part on a detected level of fluid in the collection canister.

In any of the systems described herein may include a suction unit that includes a collection canister, a vacuum pump configured to provide a negative pressure to the collection canister, and a vacuum release valve configured to release the negative pressure from the collection canister. The system may also include a controller configured to operate the vacuum pump in a first operating mode where the vacuum release valve in response to a level of fluid within the collection canister and a second operating mode where the collection canister is configured to release the negative pressure when decoupled from the vacuum pump.

In any of the systems described herein, the collection canister may include a level detector configured to determine the level of fluid within the collection canister. In some examples, the level detector may be configured to determine whether the level of fluid is greater than a predetermined threshold.

In any of the systems described herein, the suction unit may include a proximity sensor and the controller is further configured to operate the vacuum pump based on a signal from the proximity sensor. In some examples, the collection canister may be configured to receive fluid from a connecting tube in response to the negative pressure.

Any of the systems described herein may include a connecting tube, a collection unit, and a suction unit. The collection unit may include a collection canister coupled to the connecting tube and a collection bag coupled to the collection canister. The suction unit may include a controller configured to control negative pressure to the collection canister from an external negative pressure source based at least in part on a level of fluid in the collection canister.

Any of the methods described herein may include receiving, from an external negative pressure source, a negative pressure to a rigid collection canister, collecting, in response to the negative pressure, liquid from a connecting tube coupled to the rigid collection canister, wherein the liquid is collected in the rigid collection canister, and releasing the negative pressure from the rigid collection canister, wherein the release of the negative pressure causes liquid to flow from the rigid collection canister to a collection bag.

As mentioned above, the apparatuses (e.g., devices, systems, etc.) and methods described herein may be used to collect any fluid or liquid, including, but not limited to urine. Other fluids include other bodily fluids, such as mucus, liquid fecal matter, etc. The examples described herein illustrate the collection of urine; one of skill in the art would understand that these apparatuses and methods may be used to collect other liquids.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows an example fluid collection system.

FIG. 2 shows another example fluid collection system.

FIG. 3 shows an example exploded view of a suction/controller unit.

FIG. 4 shows a partial exploded view of an example fluid collection system.

FIG. 5 shows another example of a fluid collection system.

FIG. 6 shows another example of a fluid collection system.

FIGS. 7A-7J show example external urinary catheters that may be used by any fluid collection system described herein.

FIG. 8 is a block diagram of an example fluid collection system.

FIG. 9 is a flowchart showing an example method for collecting a liquid.

FIG. 10 shows a block diagram of a device that may be an example of any partial or complete fluid collection apparatus (e.g., devices or systems) described herein.

FIGS. 11A-11C schematically illustrate the operation of one example of a fluid collection apparatus including a valving sub-system when applying suction. FIG. 11A shows a first example of the valving sub-system (using a suction pump), FIG. 11B shows a second example of the valving sub-system (using wall suction/vacuum), and FIG. 11C shows the canister and collection bag during the application of suction. In general, any of the methods and apparatuses (e.g., systems) described herein may be configured to operate with an external source of vacuum, such as “wall suction” commonly available in hospitals and health centers.

FIGS. 12A-12C schematically illustrate the operation of one example of a fluid collection apparatus including a valving sub-system after suction has been turned off by triggering of a fluid level sensor. FIG. 12A shows a first example of the valving sub-system (using a suction pump), FIG. 12B shows a second example of the valving sub-system (using wall suction/vacuum) and FIG. 12C shows the canister and collection bag after suction has been turned off.

FIGS. 13A-13C schematically illustrate the operation of one example of a fluid collection apparatus including a valving sub-system after suction has been turned off but while siphoning is occurring. FIG. 13A shows a first example of the valving sub-system (using a suction pump), FIG. 13B shows a second example of the valving sub-system (using wall suction/vacuum) and FIG. 13C shows the canister and collection bag after suction has been turned off while siphoning is occurring.

### DETAILED DESCRIPTION

In general, the apparatuses described herein are configured to collect liquids, including but not limited to urine, from a patient. In some cases, the patient may be bedridden and/or incontinent, including unconscious, mobility-impaired or comatose patients. Thus, described herein are apparatuses (systems, devices, etc.) and methods of collecting and managing fluids, and in particular, urine excreted by the patient. Such apparatuses may assist in helping any patients void and remove urine from their body when they may otherwise be able to.

An apparatus for collecting fluid may generally include a suction unit, a fluid collection reservoir, and a connecting tube. The suction unit may include a vacuum pump capable of producing or generating a negative pressure or such negative pressure can be generated and/or provided by an external system, like central vacuum system in a hospital, and be supplied to the suction unit using an appropriate connection. The suction unit may be coupled to the collection lumen. In turn, the collection lumen may be coupled or attached to the patient through, in some cases, an external urinary catheter designed to collect urine from the patient. The negative pressure may be coupled to the collecting tube to draw and collect urine from the patient.

The suction unit may include a collection canister that can hold the collected fluid (e.g., urine). The collection bag may be directly or indirectly coupled to the collection canister. Fluid that has been collected and held within the collection canister may be transferred to the fluid collection reservoir. In some examples, the fluid collection reservoir may be a flexible transparent or translucent bag with calibrated markings on the sides. The markings may allow easy and accurate measurement of excreted fluid (e.g., urine or other fluids). As used herein the term fluid may refer to urine or any other liquid material.

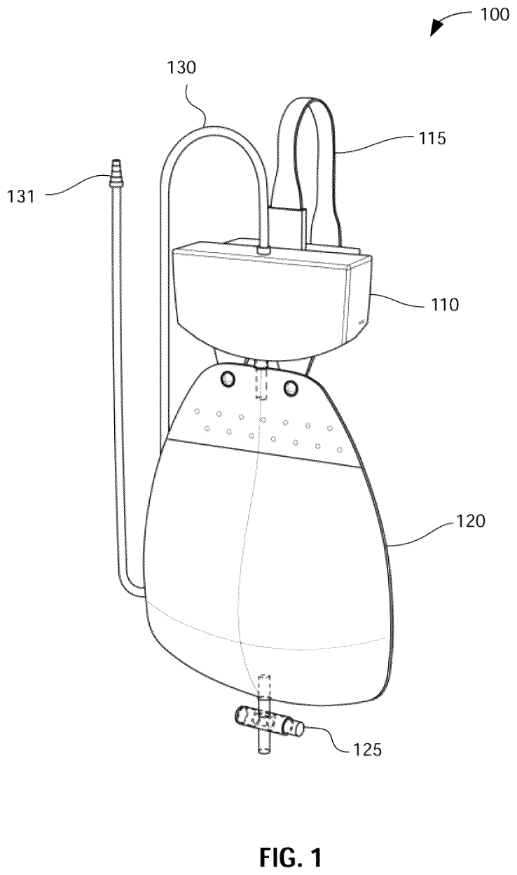

FIG. 1 shows an example fluid collection system 100. The fluid collection system 100 may include a suction unit 110, a collection bag 120, and a connecting tube 130. The fluid collection system 100 may be coupled to a patient via the connecting tube 130. The suction unit 110 may provide negative pressure (e.g., at least a partial vacuum) to the connecting tube 130. A distal end of the connecting tube 130 may be coupled to an external urinary catheter that may be positioned on a patient. Urine, or other fluids, may be collected through the connecting tube 130 and held within the suction unit 110. Urine or other fluids may also be transferred from the suction unit to the collection bag 120.

The suction unit 110 may be detachably coupled to a first or proximal end of the connecting tube 130. The connecting tube 130 may be any feasible elongate tube. For example, the connecting tube 130 may be implemented as a flexible vinyl tube, a flexible rubber tube, a semi-rigid tube, or the like. In some examples, the connecting tube 130 may consist of a plurality of individual connecting tubes that are connected together. A second or distal end of the connecting tube 130 may include a universal connector 131. The universal connector 131 may be used to couple the second end of the connecting tube 130 to any of a variety of external urinary catheters (not shown). Thus, any of the urinary catheters may be detachably coupled to the connecting tube 130. The urinary catheters may be disposed next or adjacent to the patient's urethra to collect urine from the patient.

The suction unit 110 may provide negative pressure to the connecting tube 130 in order to draw and collect fluid from the patient. After the fluid is collected by the suction unit 110, the fluid is transferred to the collection bag 120.

The collection bag 120 may be used to hold the collected fluid. In some examples, the collection bag 120 may be clear or translucent and include markings to indicate how much fluid has been collected. In some examples, the collection bag 120 may be detachably coupled to the suction unit 110 using any feasible means. In some variations, the collection bag 120 may include one or more attachment holes that may couple to corresponding pins, pegs or hooks on the suction unit 110.

In some examples, a drain valve 125 may be disposed at or toward a bottom of the collection bag 120. The drain valve 125 may be used to empty fluid from the collection bag 120 after fluid has been collected. The collection bag 120 may be disposable. Thus, after the collection bag 120 has been filled and the amount of contained fluid has been noted or recorded, the collection bag 120 may be detached from the suction unit 110 and disposed.

The suction unit 110 may include one or more mounts 115 that may be used to secure the fluid collection system 100 to a stand, bed wall, etc. The mounts 115 may include any feasible straps, clips, buckles, or the like.

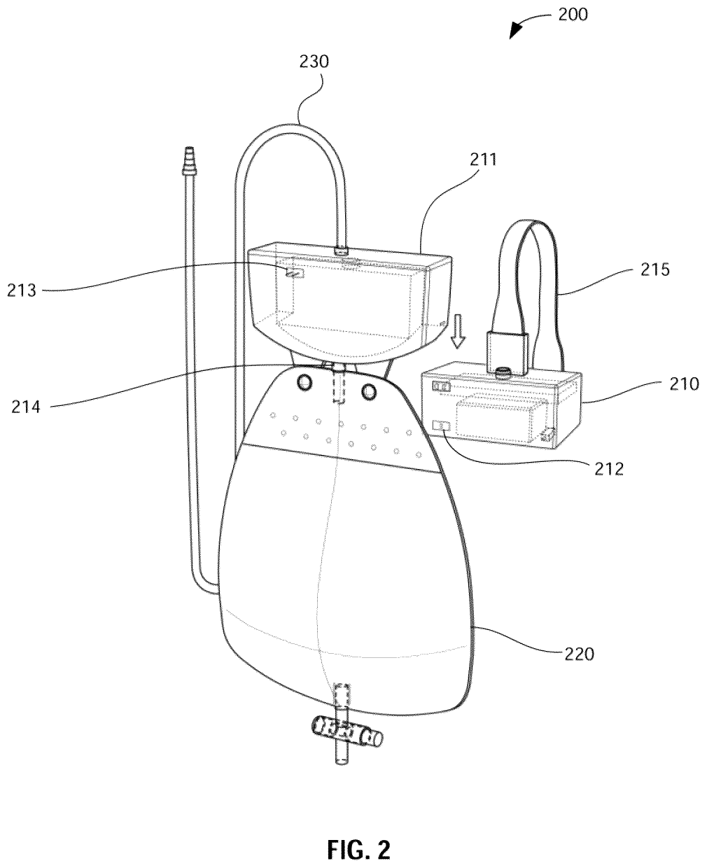

In some examples, the suction unit 110 may include an intermediate collection canister and a separable vacuum pump. One such example is described below with respect to FIG. 2.

FIG. 2 shows another example fluid collection system 200. The fluid collection system 200 may include a suction/controller unit 210, a collection canister 211, a collection bag 220, and a connecting tube 230. The collection bag 220 and the connecting tube 230 may be examples of the collection bag 120 and the connecting tube 130 of FIG. 1.

The suction/controller unit 210 may include a vacuum pump and a controller board (not shown). Although referred to herein as a vacuum pump, any other technically feasible device capable of generating or providing a negative pressure may be used. For example, any feasible suction pump, displacement pump or the like may be used. In some examples, the negative pressure may be provided by any feasible external source, such as a hospital central vacuum system. The controller board may control operations of the fluid collection system 200, such as running the vacuum pump and controlling the vacuum delivered to the collection canister 211. The suction/controller unit 210 may be powered by an included battery (not shown) or powered from an external power source. In some examples, the external power source, which may be an alternating current (AC) or direct current (DC) power source, may power the suction/controller unit 210 and charge the included battery simultaneously.

The suction/controller unit 210 and the collection canister 211 may perform equivalent or similar operations with respect to the suction unit 110 of FIG. 1. That is, the suction/controller unit 210 may provide negative pressure to the connecting tube 230 to draw urine or other fluids from the patient. The suction/controller unit 210 may be detachably coupled to the collection canister 211. The collection canister 211 may be rigid in contrast to the collection bag 220. In some examples, the vacuum pump of the suction/controller unit 210 may be coupled to the collection canister 211 as the suction/controller unit 210 is inserted into, or otherwise coupled to, the collection canister 211. The connecting tube 230 may be coupled to the collection canister 211. In this manner, negative pressure from the vacuum pump may be provided to the connecting tube 230 through the collection canister 211.

In some examples, the suction/controller unit 210 may include a proximity detector 212 to determine when the suction/controller unit 210 is inserted and/or coupled to the collection canister 211. The controller board may control operation of the vacuum pump, or application of externally provided negative pressure, based on the proximity detector 212. For example, the proximity detector 212 may enable the vacuum pump when the proximity detector 212 indicates that the suction/controller unit 210 is coupled to the collection canister 211. Examples of the proximity detector 212 may include micro-switches, hall-effect switches, optical switches, magnetic switches, or the like.

The collection canister 211 may include pegs, hooks, or the like to detachably couple and support the collection bag 220. Thus, the collection bag 220 may include one or more attachment holes corresponding to the one or more pins, pegs, hooks, etc. The collection canister 211 may also include a one-way valve 214 that couples the collection canister 211 to the collection bag 220. The one-way valve 214 may allow fluid and/or gasses to flow from the collection canister 211 to the collection bag 220, but not from the collection bag 220 to the collection canister 211.

The collection canister 211 may also include a fluid level detector 213. The fluid level detector 213, may be electrically coupled to the controller board in the suction/controller unit 210 when the suction/controller unit 210 is inserted and/or coupled to the collection canister 211. The fluid level detector 213 may indicate an amount of liquid that may be held within the collection canister 211. The fluid level detector 213 may be any feasible mechanical or electro-mechanical device capable of detecting the fluid level within the collection canister 211.

In a first operating mode, the suction/controller unit 210 may provide negative pressure through the collection canister 211 to the connecting tube 230 to collect fluids (including but not limited to urine) from the patient. The urine and/or other fluids are accumulated within the collection canister 211. In some examples, the negative pressure may also hold the fluids within the collection canister 211 by not allowing the fluids to overcome flow through the one-way valve 214. When the fluid level detector 213 indicates that the urine or other fluids exceeds a predetermined threshold, the suction/controller unit 210 may stop the vacuum pump and allow fluids from the collection canister 211 to flow through the one-way valve 214 into the collection bag 220. In some examples, gravity may assist the flow of fluids through the one-way valve. After the fluid is transferred, the fluid level detector 213 may indicate that the fluid in the collection canister 211 is less than the predetermined threshold and the controller board can restart the vacuum pump. Then, negative pressure may be again provided to the collection canister 211 and the connecting tube 230. The one-way valve 214 may prevent fluid or gas flow from the collection bag 220 to the collection canister 211, particularly when a negative pressure is provided to the collection canister 211.

In a second operating mode, the suction/controller unit 210 may provide negative pressure through the collection canister 211 to the connecting tube 230 to collect urine or other fluids from the patient. The negative pressure may be maintained until the suction/controller unit 210 is decoupled from the collection canister 211 and the collection bag 220. Loss of the negative pressure allows fluid in the collection canister 211 to pass through the one-way valve 214 into the collection bag 220. After the fluid is transferred, the suction/controller unit 210 may be reinserted or attached to the collection canister 211 and negative pressure again provided.

The suction/controller unit 210 may include a mount 215 that may an example of the mount 115 of FIG. 1. When the suction/controller unit 210 is coupled to the collection canister 211, the mount 215 can support the entire fluid collection system 200.

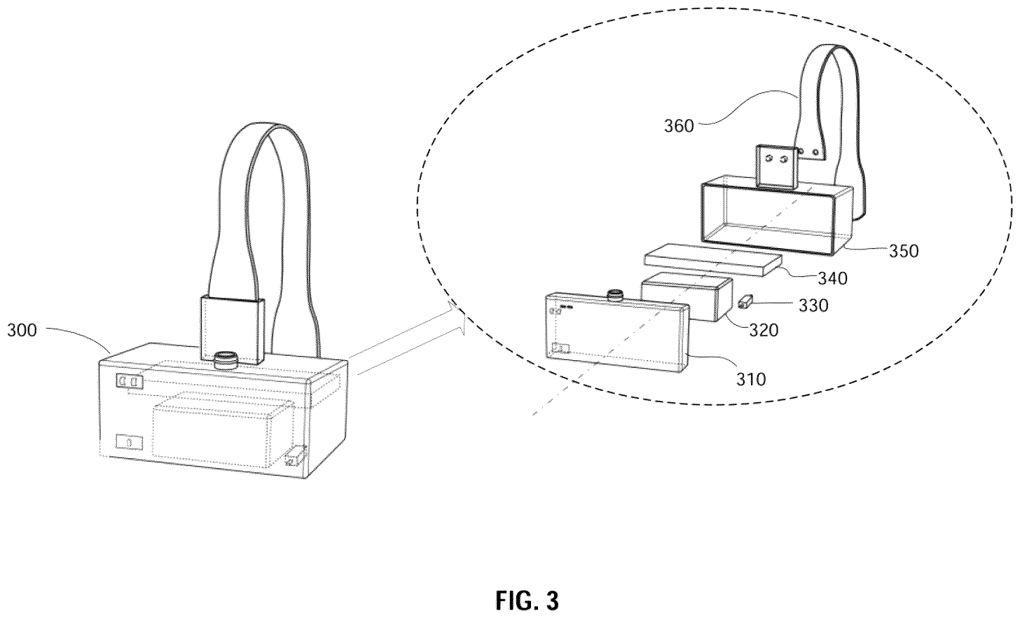

FIG. 3 shows an example exploded view of a suction/controller unit 300. The suction/controller unit 300 may be an example of the suction/controller unit 210 of FIG. 2. The suction/controller unit 300 may include a manifold 310, a vacuum pump 320, vacuum release valve 330, a controller board 340, a housing 350, and a mount 360.

The vacuum pump 320 may generate and/or provide a negative pressure (e.g., at least a partial vacuum with respect to local atmospheric pressure). The negative pressure may be coupled from the vacuum pump 320 to the manifold 310. In some other examples, the negative pressure may be provided by an external vacuum source, such has a central vacuum system. The manifold 310 may be coupled to the collection canister (not shown) when the suction/controller unit 300 is coupled to a collection canister, such as the collection canister 211 of FIG. 2. The vacuum release valve 330 may be normally closed. For example, when the vacuum release valve 330 is not activated, a negative pressure may be established and or maintained within the collection canister 211 and/or the manifold 310. On the other hand, when the vacuum release valve 330 is activated, then the negative pressure may be vented to the atmosphere. Venting the negative pressure may dissipate any negative pressure that may be present in the manifold 310 and in the collection canister 211, which may be coupled to the manifold 310.

The controller board 340 may control operations of the suction/controller unit 300. For example, the controller board 340 may be coupled to and operate the vacuum pump 320 and the vacuum release valve 330. In some examples, the controller board 340 may operate the vacuum pump 320 and the vacuum release valve 330 in a first operating mode. Thus, the vacuum pump 320 may be enabled and the vacuum release valve 330 operated not to vent negative pressure. In the first operating mode, the controller board 340 may control the vacuum pump 320 and the vacuum release valve 330 based on a detected fluid level in the collection canister 211. For example, the fluid level detector 213 within the collection canister 211 may be coupled to the controller board 340. The controller board 340 can control the vacuum pump 320 and vacuum release valve 330 to provide a negative pressure based on a determined fluid level.

In another example, the controller board 340 may operate the vacuum pump 320 and the vacuum release valve 330 in a second operating mode. In the second operating mode, the controller board 340 can operate the vacuum pump 320 and the vacuum release valve 330 based on determining that the suction/controller unit 300 is inserted into the collection canister 211. For example, a proximity detector, electrically coupled to the controller board 340, may determine whether the suction/controller unit 300 is inserted or affixed to the collection canister 211. The controller board 340 can control the vacuum pump 320 and vacuum release valve 330 to provide a negative pressure based on a determination that the suction/controller unit 300 inserted or affixed to the collection canister 211.

The housing 350 may provide a means to mount, house, and/or protect components of the suction/controller unit 300. In some examples, the manifold 310, the vacuum pump 320, the vacuum release valve 330, and the controller board 340 may be housed within the housing 350. The mount 360, which may be an example of the mount 115 of FIG. 1, may be coupled to the housing 350.

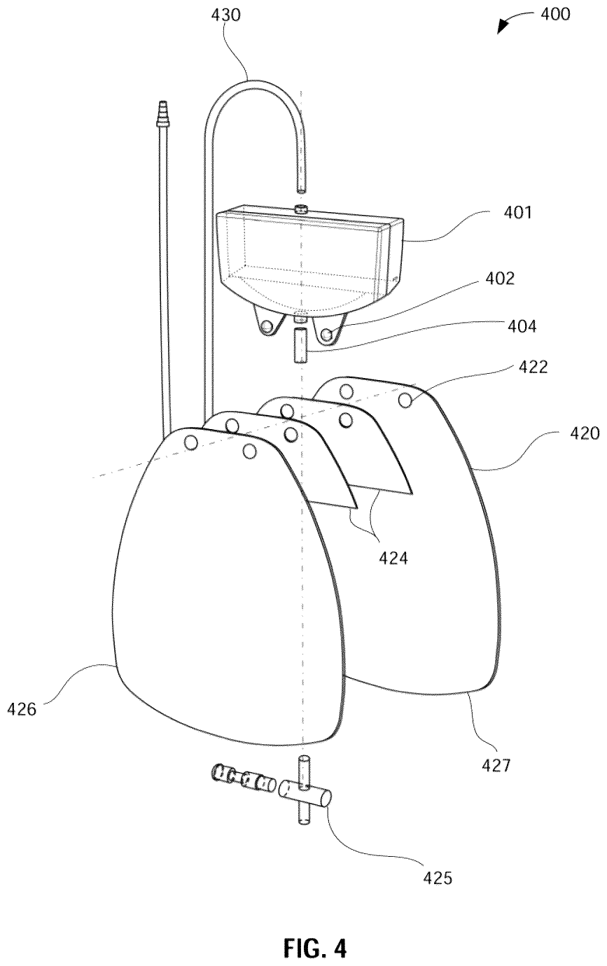

FIG. 4 shows a partial exploded view of an example fluid collection system 400. The fluid collection system 400 may include a collection canister 401 and collection bag 420. The collection canister 401 can be an example of the collection canister 211 of FIG. 2 and the collection bag 420 can be an example of the collection bag 220 of FIG. 2.

The collection canister 401 may include one or more mounting pins 402. The mounting pins 402 may engage with one or more attachment holes 422 included within the collection bag 420. In this manner, the collection bag 420 may be supported and/or hang from the collection canister 401. In some examples, a tube 404 may couple the collection canister 401 to the collection bag 420. The tube 404 may optionally include a one-way valve to prevent the flow of fluid and/or gasses from the collection bag 420 to the collection canister 401. A connecting tube 430 may be coupled to an upper portion of the collection canister 401. The connecting tube 430 may be an example of the connecting tube 130 of FIG. 1 and/or the connecting tube 230 of FIG. 2.

In some examples, the collection bag 420 may include a first side 426, a second side 427, and a one-way valve 424. The one-way valve 424 may be used alternatively or in addition to a one-way valve that may be included within the tube 404. The one-way valve 424 may be formed from two or more sheets of material. The first side 426, the second side 427, and the one-way valve 424 may be a flexible, conformable material such as vinyl, rubber, or the like. In some examples, the collection bag 420 may include a drain 425 that may be used to empty contained fluids.

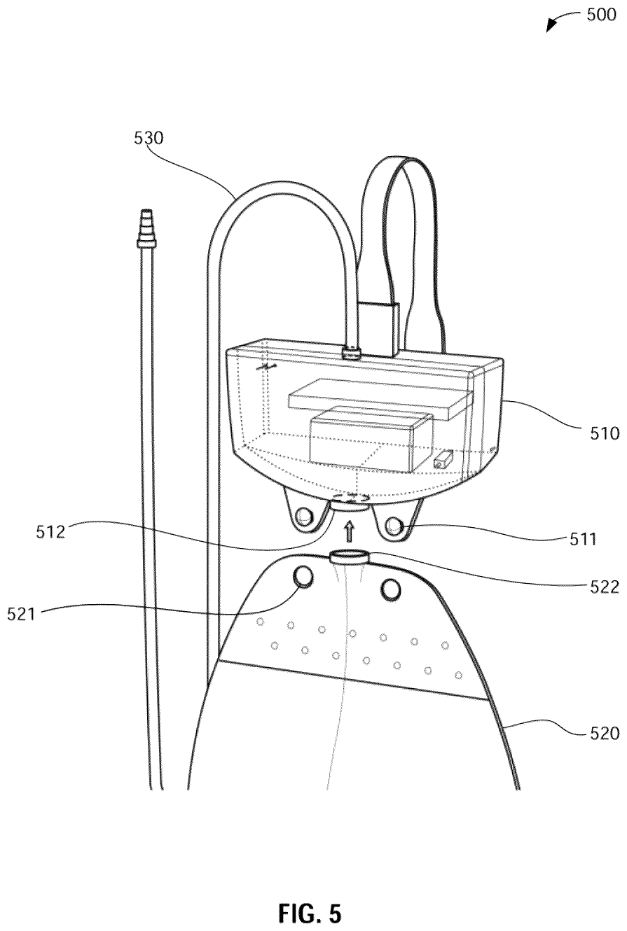

FIG. 5 shows another example of a fluid collection system 500. The fluid collection system 500 may include a suction unit 510 and a collection bag 520. The suction unit 510 may be a single integrated unit that includes a collection canister (such as the collection canister 211 of FIG. 2) and a suction/controller unit (such as the suction/controller unit 210 of FIG. 2). Thus, the suction unit 510 may include a vacuum pump, a controller board, a battery, and/or other components that may be included with the suction/controller unit 210. A connecting tubing 530 may couple the suction unit 510 to a patient's external urinary catheter.

In some examples, the suction unit 510 may be used with many patients while the collection bag 520 may be disposable. The suction unit 510 may include one or more mounting pins 511 that may engage with one or more attachment holes 521 included in the collection bag 520. In this manner, the mounting pins 511 may hold and/or support the collection bag 520. The suction unit 510 may also include an output port 512. Similarly, the collection bag 520 may include an input port 522. When the collection bag 520 is connected to the suction unit 510, the output port 512 may be coupled to the input port 522. The output port 512 may be connected to a collection canister within the suction unit 510. In this manner, fluids collected through the connecting tubing 530 and held in a collection canister may be transferred to the collection bag 520. In some examples, the collection bag 520 may be an example of the collection bag 120 of FIG. 1. Therefore, the collection bag 520 may include a drain valve (not shown).

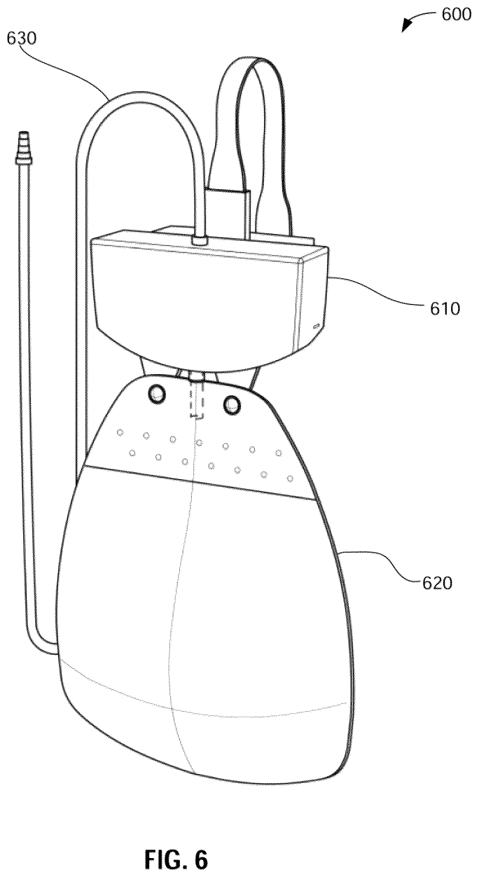

FIG. 6 shows another example of a fluid collection system 600. The fluid collection system 600 may include a suction unit 610, a collection bag 620, and connecting tubing 630. The suction unit 610 may be similar to a combination of the suction/controller unit 210 and collection canister 211 of FIG. 2. The collection bag 620 may be similar to the collection bag of 220 of FIG. 2, however the collection bag 620 may not include a drain valve 125.

In some examples, the collection bag 620 may be permanently affixed or otherwise coupled to the collection reservoir of the suction unit 610. Thus when the collection bag 620 becomes full, then the collection bag 620 and the collection reservoir may be discarded. However, the suction/controller unit may be reused.

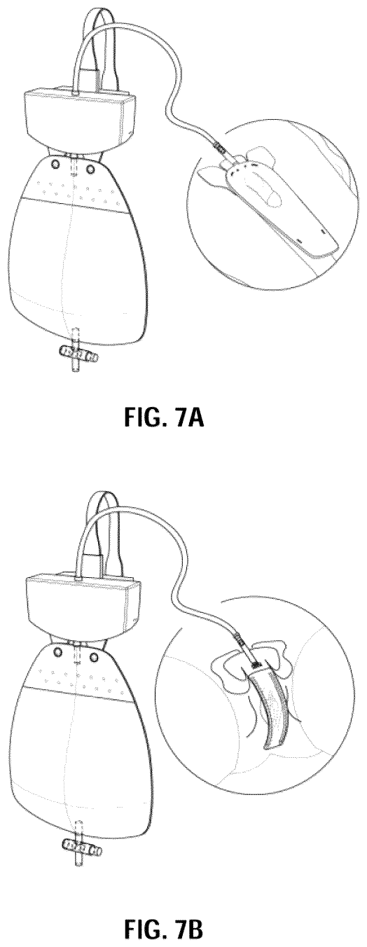

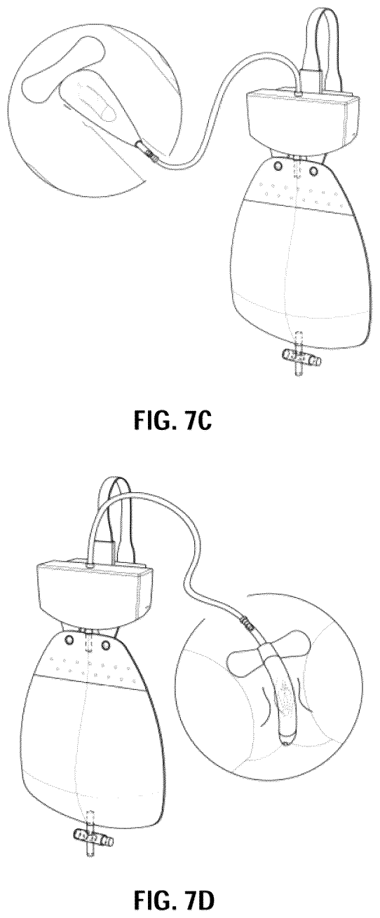

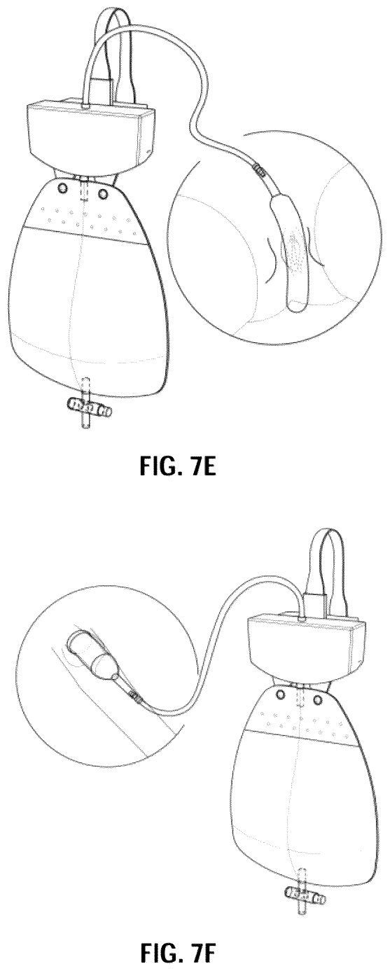

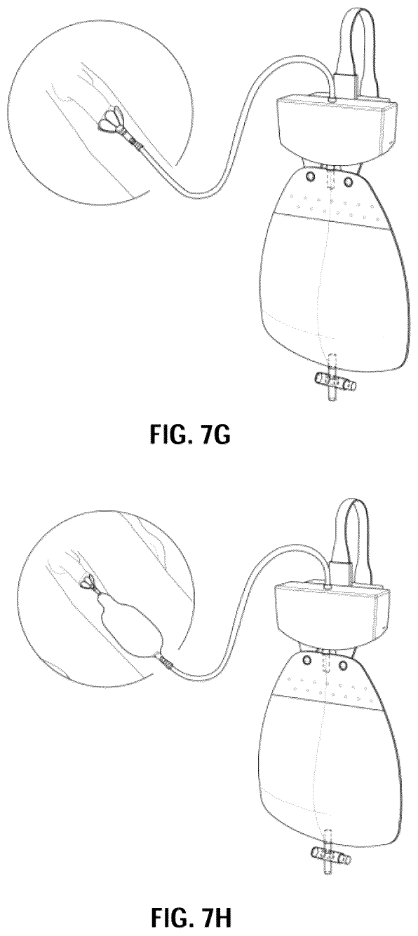

FIGS. 7A-7J show example external urinary catheters that may be used by any fluid collection system described. As described above, a connecting tube may be coupled to a variety of external urinary catheters that may be used to collect urine and/or other fluids from patients. Example external urinary catheters may include, without limitation, Consure Medical QiVi male and female external catheters, Sage PrimoFit male and Sage PrimaFit female external catheters, BD PureWick external catheters, Men's Liberty external catheters, condom catheters, TillaCare UriCap female external catheters, and VOCA male and female external drainage bags. Still other external catheters not listed here may be coupled to the connecting tube.

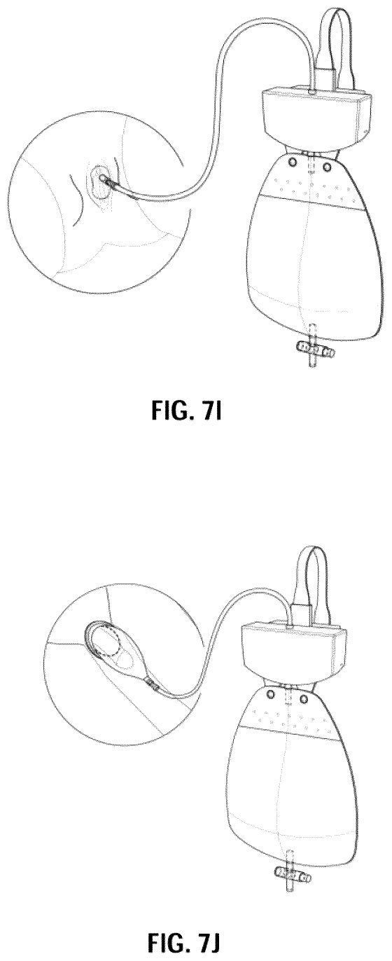

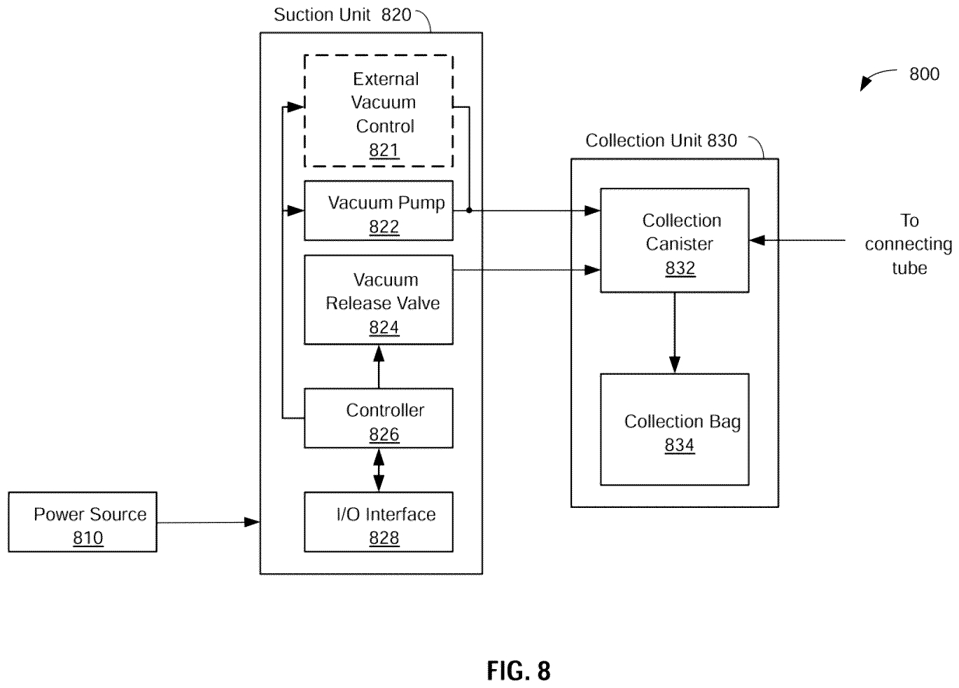

FIG. 8 is a block diagram of an example fluid collection system 800. The fluid collection system 800 may include a power source 810, a suction unit 820, and a collection unit 830. The power source 810 may be an external AC or DC power source that may be coupled and provide power to devices within the suction unit 820. In some examples, the suction unit 820 may include a battery (not shown) that may provide power alternatively or in addition to the power source 810.

The suction unit 820 may include a vacuum pump 822, a vacuum release valve 824, a controller 826, and an input/output (I/O) interface 828. The vacuum pump 822 may provide or generate a negative pressure or vacuum. The vacuum pump 822 may be coupled to and controlled by the controller 826. The suction unit 820 may also include an optional external vacuum control unit 821 (optional components are depicted with dashed lines.) The vacuum control unit 821 may receive negative pressure from an external source, such as a central vacuum system. The controller 826 may control the negative pressure provided from an external source.

The collection unit 830 may include a collection canister 832 and a collection bag 834. The collection canister 832 may receive the negative pressure or vacuum from the vacuum pump 822 or the vacuum control unit 821. The collection canister 832 may be coupled to a connecting tube that, in turn, may be coupled to an external urinary catheter. In this manner, the negative pressure or vacuum from the vacuum pump 822 may draw urine or other fluids from the patient into the collection canister 832. The collection canister 832 may include a level detector that can determine when collected fluids exceed a predetermined threshold amount. The level detector may be coupled to the controller 826. The collection bag 834 may be coupled to the collection canister 832 through a one-way valve.

The controller 826 may be electrically coupled to the vacuum pump 822, the vacuum release valve 824 and the level detector of the collection canister 832. In some examples, the controller 826 may be a controller board and include one or more processors and memory.

The controller 826 may operate the fluid collection system 800 in a first operating mode where the vacuum pump 822 provides a negative pressure to the collection canister 832 based at least in part on whether the level of liquid in the collection canister 832 exceeds a predetermined threshold. The controller 826 may also operate the fluid collection system 800 in a second operating mode where the vacuum pump 822 provides a negative pressure to the collection canister 832 until the negative pressure is lost by moving, removing, or disconnecting the collection canister 832 from the vacuum pump 822.

The I/O interface 828 may include any feasible switches, displays, light emitting diodes (LEDs) or the like. For example, the I/O interface 828 may include devices or circuits to interface with a level detector such the fluid level detector 213 of FIG. 2. In another example, the I/O interface 828 may include devices or circuits to interface with a proximity detector, such as the proximity detector 212.

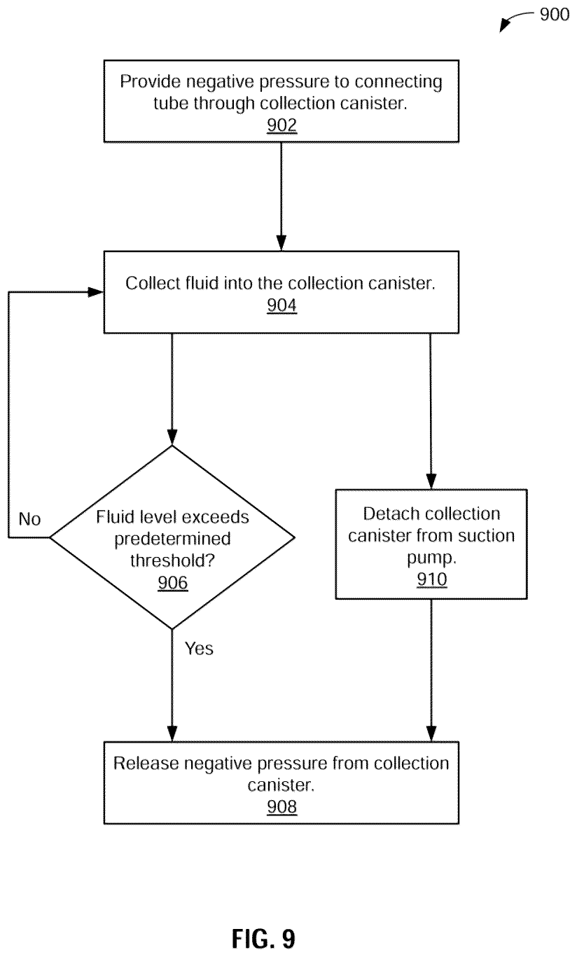

FIG. 9 is a flowchart showing an example method 900 for collecting a liquid. Some examples may perform the operations described herein with additional operations, fewer operations, operations in a different order, operations in parallel, and some operations differently. The method 900 is described below with respect to the fluid collection system 200 of FIG. 2, however, the method 900 may be performed by any other suitable system or device.

The method 900 begins in block 902 as the suction/controller unit 210 provides negative pressure to the connecting tube 230 through the collection canister 211. For example, a vacuum pump within the suction/controller unit 210 may be coupled to the collection canister 211. The connecting tube 230 may also be coupled to the collection canister 211. As the vacuum pump provides negative pressure to the collection canister 211, the negative pressure may also be provided to the connecting tube 230.

Next, in block 904 urine or other fluids is collected into the collection canister. For example, the collecting tube 230 may be coupled to an external urinary catheter that is disposed near a urethra. The negative pressure may draw urine or other fluids through the connecting tube and into the collection canister 211.

Next, in block 906 the suction/controller unit 210 determines whether the urine (or other fluid) level exceeds a predetermined threshold in the collection canister 211. For example, the fluid level detector 213 may determine whether the fluid level in the collection canister 211 exceeds the predetermined threshold. If the fluid level does not exceed the predetermined threshold, then the method returns to block 904. On the other hand, if the fluid level exceeds the predetermined threshold, then in block 908 the negative pressure is released from the collection canister 211. Releasing the negative pressure may enable liquid collected in the collection canister 211 to flow into the collection bag 220. The method returns to block 902. In some examples, the negative pressure may be released as the suction/controller unit 210 operates a vacuum release valve such as the vacuum release valve 330 of FIG. 3. In some examples, the suction/controller unit 210 may perform the operations associated with block 906 when operating in a first operating mode.

Returning to block 904, alternatively or in addition to proceeding to block 906, the method proceeds to block 910 where the collection canister 211 is detached from the vacuum pump. The method proceeds to block 908. Detaching the collection canister 211 from the vacuum pump (in block 910) releases the negative pressure from the collection canister 211 (in block 908).

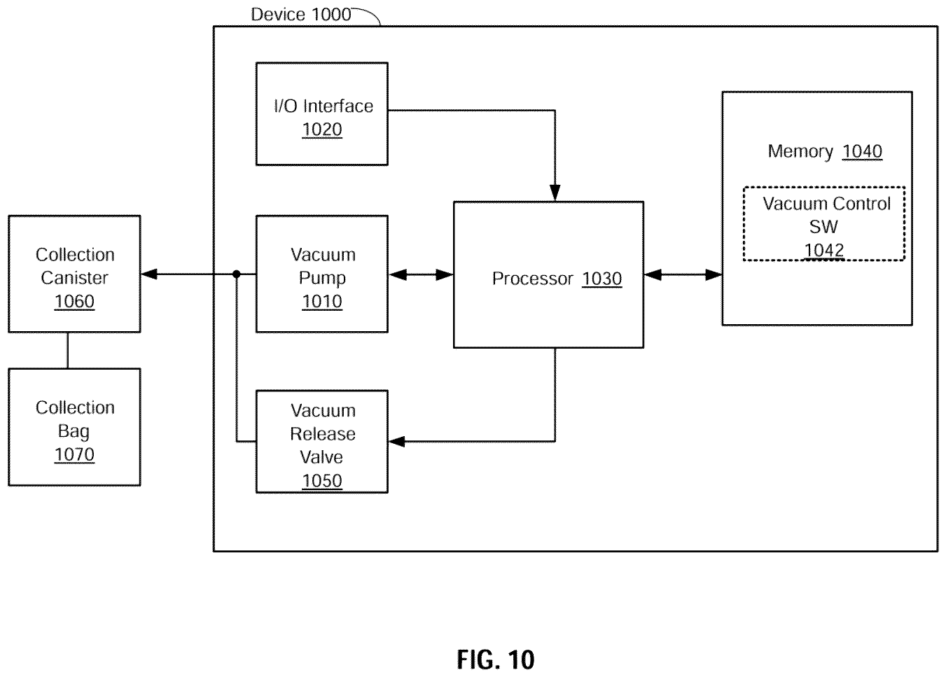

FIG. 10 shows a block diagram of a device 1000 that may be an example of any partial or complete fluid collection apparatus (e.g., devices or systems) described herein. The fluid collection device 1000 may include a vacuum pump 1010, an I/O interface 1020, a processor 1030, and a memory 1040.

The I/O interface 1020, which is coupled to the processor 1030, may be used to interface with any feasible fluid level detectors, proximity detectors, switches, LEDs or the like.

The vacuum pump 1010, which is also coupled directly or indirectly to the processor 1030, may be used generate or provide a negative pressure. In some examples, the vacuum pump 1010 may be coupled to a collection canister 1060. The collection canister 1060 may be coupled to a connecting tube which, in turn, may be coupled to an external urinary catheter. Thus, negative pressure from the vacuum pump 1010 may draw urine or other fluids through the connecting tube and into the collection canister 1060.

The vacuum release valve 1050 may be coupled to the processor 1030 and directly or indirectly to the collection canister 1060. In some examples, the processor 1030 may operate the vacuum release valve 1050 to release the negative pressure from the collection canister 1060. Releasing the negative pressure may enable or allow fluid to flow from the collection canister 1060 to a collection bag 1070.

The processor 1030, which is also coupled to the memory 1040, may be any one or more suitable processors, micro-controllers, state machines, or the like capable of executing scripts or instructions of one or more software programs stored in the device 1000 (such as within the memory 1040).

The memory 1040 may include a non-transitory computer-readable storage medium (e.g., one or more nonvolatile memory elements, such as EPROM, EEPROM, Flash memory, a hard drive, etc.) that may store a vacuum control software module 1042. The vacuum control software module 1042 includes program instructions that, when executed by the processor 1030, may cause the device 1000 to perform the corresponding function(s). Thus, the non-transitory computer-readable storage medium of memory 1040 may include instructions for performing all or a portion of the operations described herein.

The processor 1030 may execute the vacuum control software module 1042 to control vacuum operations. For example, execution of the vacuum control software module 1042 may enable the processor 1030 to determine a fluid level in the collection canister 1060. Then, based on the determined fluid level, the processor 1030 may operate the vacuum release valve 1050 to release any negative pressure from the collection canister 1060. In some examples, execution of the vacuum control software module 1042 may determine, using a proximity detector, whether the collection canister 1060 is coupled to the device 1000. If the collection canister 1060 is coupled, then the processor 1030 may operate the vacuum pump 1010.

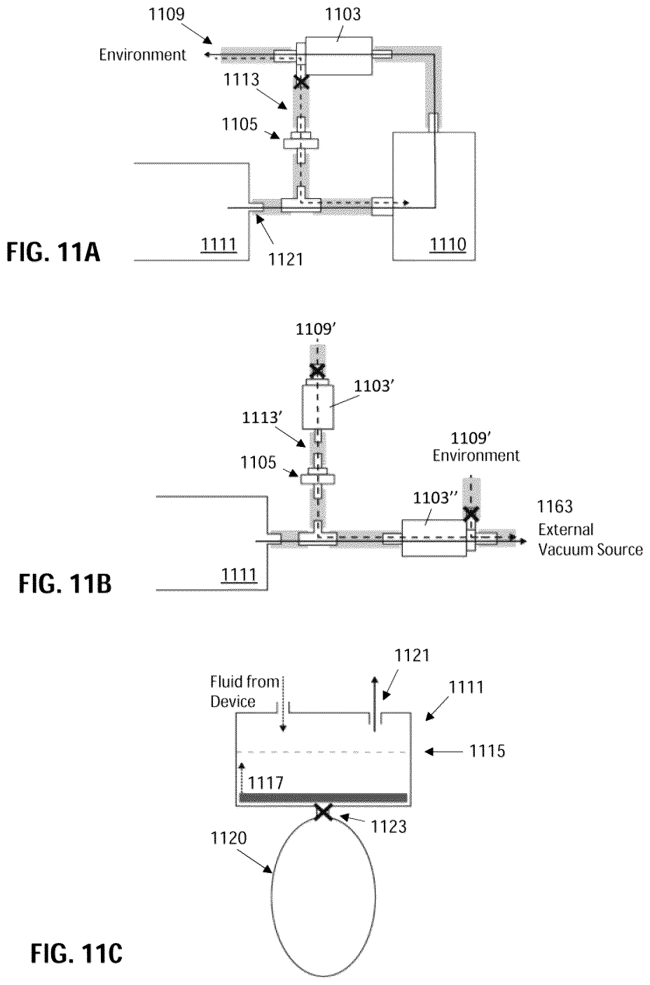

As mentioned above, any of the methods and apparatuses (e.g., systems, devices, etc., including urine collection systems) described herein may include a valving sub-system that may be configured to limit or prevent backflow. For example any of these apparatuses may include a one or more valves and bypass lines between the canister and the suction pump (or wall suction source) to prevent or limit the flow of fluid from the canister into the suction source either during the application of suction or even when suction is turned off, including when siphoning may occur. For example, FIGS. 11A-11C, 12A-12C and 13A-13B illustrate one example of valving sub-system configured to prevent backflow that may be included as part of any of these apparatuses. FIG. 11A schematically illustrates the components of the valving sub-system connected between the collection canister 1111 and a suction pump 1110, including a three-way solenoid valve 1103 that is configured to prevent the flow of fluid between the suction pump 1110 and an open air inlet 1109. The inlet 1109 may be open to the air (e.g., environment) and/or may be coupled to an odor filter (not shown).

In FIG. 11A a first example of a system is shown with a suction pump 1110 on, and the three-way solenoid valve 1103 off, so that the open air inlet 1109 is connected to the suction pump 1110 outlet. In the off state the three-way solenoid valve 1103 does not connect the open air inlet 1109 to a bypass line 1113 that otherwise connects the suction port 1121 of the collection canister 1111 to the open air inlet via a T-junction before the suction pump 1110 is connected to the collection canister inlet. The bypass line 1113 also includes a one-way valve 1105 arranged to allow the flow of air from the open air inlet 1109 to the canister suction port 1121, but to prevent air from flowing into the canister inlet or fluid from flowing out of the canister inlet and out of the open air inlet. In the configuration shown in FIG. 11A the suction pump 1110 applies a negative pressure within the collection canister 1111 via the canister suction port 1121; air flow passes from the canister suction port through the suction pump 1110 to the solenoid valve 1103 and out of the open air inlet 1109. When the suction pump is on, the three-way solenoid valve 1103 is off, allowing flow from the suction pump out of the open air inlet 1109. The three-way solenoid closes the connection between the bypass line 1113 and the open air inlet 1109, so that air cannot flow through the bypass line as long as the three-way solenoid valve 1103 is off. The bypass line 1113 is helpful particularly where the suction pump 1110 is configured to prevent airflow in the reverse direction (e.g., out of the inlet coupled to the canister suction port) when the suction pump is off. Thus, when the suction pump is off air can still flow through the pump in the same direction as when suction is being applied, but air cannot flow in the opposite direction.

FIG. 11B is similar to the example shown in FIG. 11A but configured for use with a wall source of suction (e.g., external vacuum source 1163). In this configuration the valving sub-system is connected between the collection canister 1111 and an external vacuum source 1163, including a three-way solenoid valve 1103″ that is configured to prevent the flow of fluid between the external vacuum source 1163 and an open air inlet 1109′. The inlet 1109′ may be open to the air (e.g., environment) and/or may be coupled to an odor filter (not shown).

In FIG. 11B, as in FIG. 11A, the three-way solenoid valve 1103″ is in the off state and does not connect the suction port 1121 of the canister 1111 to the open air inlet 1109′ via a T-junction before the source of external suction 1163 is connected to the collection canister inlet. A bypass line 1113′ also includes a one-way valve 1105 arranged to allow the flow of air from the open air inlet 1109″ to the canister suction port 1121, but to prevent air from flowing into the canister inlet or fluid from flowing out of the canister inlet and out of the open air inlet. In the configuration shown in FIG. 11B the external vacuum source 1163 applies a negative pressure within the collection canister 1111 via the canister suction port 1121. When the suction pump is on, the three-way solenoid valve 1103″ and a second valve 1103′ (on the bypass line 1113′) are both off, allowing flow from the canister and out of the external vacuum source 1163. The three-way solenoid 1103″ closes the connection between the open air inlet 1109′, and the separate valve 1103′ on the bypass line 1113′ is closed so that air cannot flow through the bypass line.

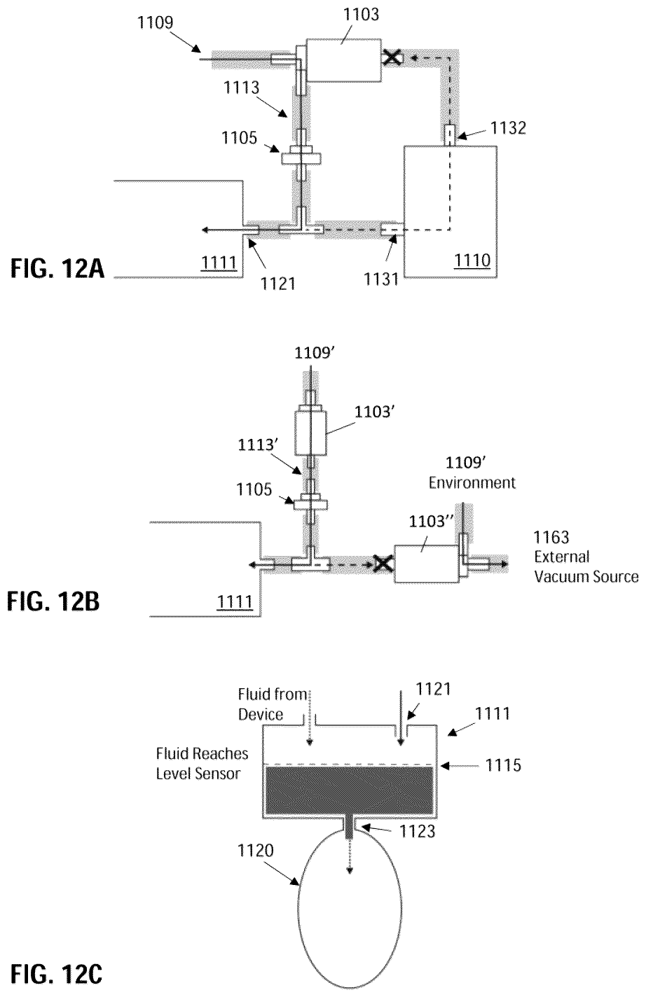

As shown in FIG. 11C, when the suction pump is on (and the three-way solenoid 1103, 1103′ is off), negative pressure is applied within the rigid or semi-rigid body of the collection canister 1111 since suction is being drawn through the suction port 1121. This draws any fluid from the urine collection device (e.g., urinary catheter), and into the collection canister. Fluid is collected within the fluid collection canister but does not normally pass into the collection bag 1120 because a pressure-based valve (e.g., a flutter valve 1123) is connected between the collection canister and the collection bag. Fluid cannot flow from the collection canister into the collection bag because the negative pressure (suction) being applied in the canister results in a low pressure in the canister as compared to the bag. Thus fluid may continue to rise within the canister 1117. The fluid level in the canister may be monitored by one or more detectors (e.g., sensors) which may be configured so that when the fluid level reaches a threshold 1115 the suction may be turned off, as shown in FIGS. 12A-12C.

The suction may be turned off when the fluid in the collection canister 1111 reaches or exceed a threshold level. Alternatively or additionally in some examples the suction pump 1110 may be turned off when after a predetermined time period, or may be turned on/off in a predetermined or user-set cycle. The any of these examples and methods the suction pump may also or alternatively be manually turned off or on.

In FIG. 12A the schematic illustrates the airflow through the valve sub-assembly in an example including a suction pump 1110, when the suction pump 1110 is off so that suction is no longer actively being applied within the canister 1111. FIG. 12B is a similar arraignment in which the source of suction is a wall suction line (as in FIG. 12B) 1163. In this case, the three-way solenoid valve is also turned on when the suction pump is turned off; in general, the system may be configured to turn the solenoid on when the suction pump is turned off, and/or turn the solenoid off when the suction pump it turned on, either immediately or after a delay. Although the suction may otherwise permit air to continue to flow in the same direction as when suction is being applied (e.g., from the pump inlet 1131 to the pump outlet 1132), because the three-way solenoid is off, flow through the suction pump is prevented.

Switching on the three-way solenoid 1103 as shown in FIG. 12A connects the canister port and therefore the chamber of the collection canister to the open air inlet 1109 through the bypass line 1113, as shown.

Similarly, FIG. 12B illustrates the configuration of the valving sub-assembly when a wall suction line (external vacuum source 1163) is used instead (or in addition to) a suction pump. In FIG. 12B when the three-way valve 1103″ in front of the source of external suction 1163 is switched off, and the second valve 1103′ is open, so that the bypass line 1113′ is open to the environment 1109′.

FIG. 12C shows the collection canister 1111 and collection bag 1120 when suction is no longer being applied, e.g., because fluid has reached the detector threshold 1115 within the collection canister. Thus, airflow from the environment is allowed to enter the collection canister via the canister port 1121, and the pressure within the collection canister increases (e.g., to atmosphere); this allows fluid to drain from the collection canister into the collection bag by gravity, since the flutter valve is allowed to open.

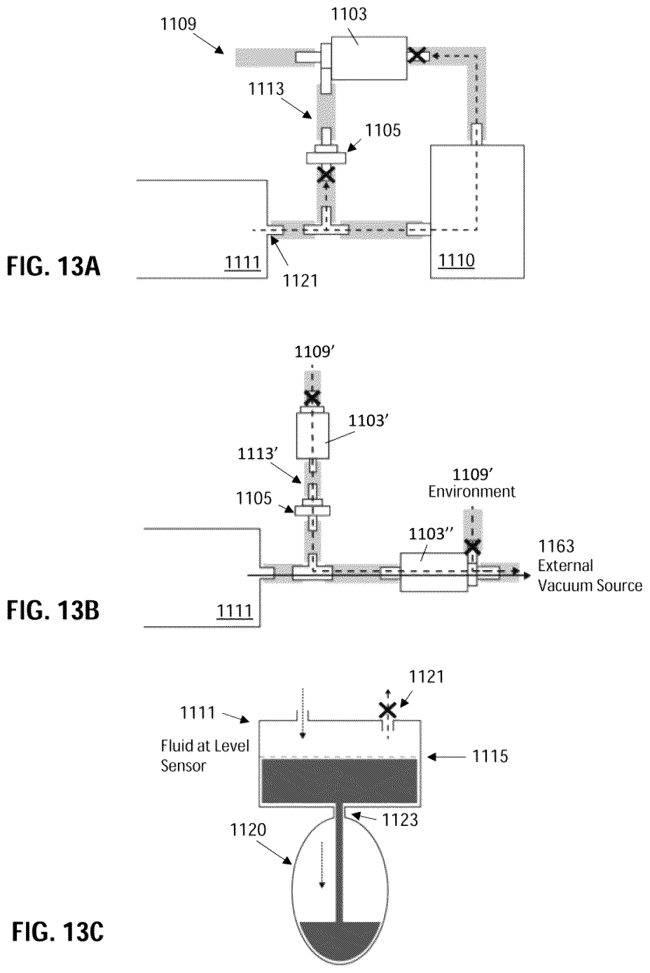

In some cases flow into the canister may continue even after the suction pump 1110 has been turned off, as illustrated in FIGS. 13A-13C. In this case, a siphon effect may occur. Thus, the chamber may continue to fill. If the rate at which the collection bag is filling from the canister is slower than the fluid being siphoned into the canister, and/or if the collection bag is full, there may otherwise be a risk of fluid passing from the suction port of the collection canister back into the suction pump and/or out of the open air inlet, which may otherwise damage the system. As shown schematically in FIG. 13A, this may be prevented by the valving sub-assembly, which prevents the flow of air or fluid from the canister suction port to the suction pump 1110 or through the bypass line 1113 and out of the open air inlet 1109 when the suction pump is off and the three-way solenoid 1103 is on. Because of the arrangement of the valves described above, air or fluid is unable to flow in either of these directions. Since fluid and air cannot flow out of the suction port in this situation, the fluid within the canister is unable to enter the suction pump or bypass line, which contain trapped air. The trapped air forms a barrier preventing fluid from the collection canister from passing through the suction port. Thus, fluid level within the canister may be maintained at a maximum level, until the rate of drainage of the fluid from the canister into the collection bag becomes equal to the rate of fluid entering the collection canister. A similar effect is seen in the arrangement in FIG. 13B, showing the system connected to a wall line of suction (e.g., external vacuum source).

While FIGS. 11A to 13B describe embodiments involving a selectively activatable suction pump (which may be switched between an off-state and an on-state), it would be understood that any other suction mechanism may be implemented in a manner consistent with the invention-including for example, a central wall suction apparatus, or an external suction apparatus which is always on, or a suction apparatus where the activation controls of such external suction apparatus are external to the systems of the present invention.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for collecting fluid from a patient, the system comprising:
a connecting tube;
a collection unit comprising:
a collection canister coupled to the connecting tube;
a collection bag coupled to the collection canister; and
a suction unit comprising:
a vacuum pump removably coupled to the collection unit and configured to provide a negative pressure to the collection canister; and
a controller configured to operate the vacuum pump based at least in part on a level of fluid in the collection canister and whether the suction unit is coupled to the collection unit.

2. The system of claim 1, wherein the collection unit is configured to provide the negative pressure to the connecting tube and collect fluid from the connecting tube in the collection canister.

3. The system of claim 1, wherein the collection bag is coupled to the collection canister through a one-way valve configured to allow fluid flow from the collection canister to the collection bag and prohibit flow from the collection bag to the collection canister.

4. (canceled)

5. (canceled)

6. The system of claim 1, wherein the suction unit includes a proximity detector configured to determine whether the suction unit is coupled to the collection unit.

7. The system of claim 1, wherein the collection canister includes a fluid level detector configured to determine a fluid level within the collection canister.

8. The system of claim 1, wherein the suction unit includes a vacuum release valve configured to release vacuum from the collection canister.

9. The system of claim 8, wherein the controller is configured to operate the vacuum release valve based on a detected level of fluid in the collection canister.

10. The system of claim 1, wherein the collection canister is configured to release, by gravity, fluid into the collection bag when vacuum is released from the collection canister.

11. (canceled)

12. (canceled)

13. (canceled)

14. The system of claim 1, further comprising a pressure valve between the collection canister and the collection bag configured so that fluid passes from the collection canister to the collection bag by gravity when the pressure within the canister is greater than the pressure within the collection bag.

15. (canceled)

16. The system of claim 1, further comprising a valve sub-system coupled to the suction unit and the collection unit and configured to prevent backflow of fluid from the collection canister into the suction unit.

17. The system of claim 1, wherein the connecting tube is configured to be coupled to at least one of a plurality of external urinary catheters.

18. A method of collecting fluid comprising:
providing, by a source of negative pressure, a negative pressure to a rigid collection canister;
collecting, in response to the negative pressure, fluid from a connecting tube coupled to the rigid collection canister, wherein the fluid is collected in the rigid collection canister; and
releasing the negative pressure from the rigid collection canister, wherein the release of the negative pressure causes fluid to flow from the rigid collection canister to a collection bag.

19. The method of claim 18, wherein the negative pressure is released by a vacuum release valve.

20. The method of claim 19, wherein the vacuum release valve is operated in response to a detected fluid level in the rigid collection canister.

21. (canceled)

22. The method of claim 18, wherein the negative pressure is released in response to decoupling the rigid collection canister from the source of negative pressure.

23. The method of claim 18, wherein the fluid flows through a one-way valve from the rigid collection canister to the collection bag.

24. (canceled)

25. A system for collecting fluid from a patient, the system comprising:
a suction unit comprising:
a collection canister;
a vacuum pump configured to provide a negative pressure to collection canister;
and
a vacuum release valve configured to release the negative pressure from the collection canister; and
a controller configured to operate the vacuum pump in:
a first operating mode wherein the vacuum release valve is operated in response to a level of fluid within the collection canister; and
a second operating mode, wherein the collection canister is configured to release the negative pressure when decoupled from the vacuum pump.

26. The system of claim 25, wherein the collection canister includes a level detector configured to determine the level of fluid within the collection canister.

27. The system of claim 26, wherein the level detector is configured to determine whether the level of fluid is greater than a predetermined threshold.

28. The system of claim 25, wherein the suction unit includes a proximity sensor and the controller is further configured to operate the vacuum pump based on a signal from the proximity sensor.

29. (canceled)

30. (canceled)

31. (canceled)
